(19) United States
(12) Patent Application Publication
(10) Pub. No.: US 2012/0071850 A1
(43) Pub. Date: Mar. 22, 2012

(54) Diaper with hand receiving pocket

(76) Inventor: Peter C. Tomassetti, Pompano Beach, FL (US)

(21) Appl. No.: 12/924,128

(22) Filed: Sep. 21, 2010

(51) Int. Cl. A61F 13/551; A61F 13/49

(52) U.S. Cl. 604/385.13; 604/385.01

(57) ABSTRACT

A diaper has a pocket on the inside back panel for placement of the installer's hand, creating a controlled mitten effect, that allows the installer to easily slide the diaper underneath a baby's bottom and into an optimal position for installation, while the installer simultaneously holds the baby's legs with the other hand to lift the baby's bottom above the changing table surface. The pocket provides a barrier between the baby and the installer's hand to protect the baby's skin from the installer's fingernails, rings, or rough hands. The pocket further serves as a means for securing a soiled diaper, which can be rolled up and folded into the pocket, allowing for easy disposal of the soiled diaper.

### BACKGROUND OF THE INVENTION

1. Field of the Invention

The present invention relates to a baby diaper and, more particularly, to a baby diaper having a pocket located on the inside back panel that allows the installer to easily slide the diaper under the baby's bottom and into the optimal position.

2. Discussion of the Related Art

A diaper is a garment customarily worn by babies during the toilet training process that is typically composed of a sponge-like material suitable for absorbing liquid. Traditional cloth diapers consisted of a folded piece of cloth fastened with safety pins. Cloth diapers have advanced to include a variety of features, such as waterproof exteriors and VELCRO fastening systems. In recent decades, however, disposable diapers have become a widely used alternative to traditional cloth diapers, as their expendable nature promotes convenience for parents and caregivers alike.

The process of installing a diaper, whether a cloth diaper or a disposable diaper, is certainly a tedious process, even for the seasoned diaper-changing veteran. A baby has sensitive skin that can be easily irritated by an installer's fingernails, rings, or rough skin while the installer attempts to slide the diaper underneath the baby and into the optimal changing position. Consequently, both the safety of the baby and the ease associated with installing a new diaper are characteristics of primary importance to parents and caregivers when selecting a diaper for their baby.

Disposing of a soiled diaper presents a similarly arduous process, as maintaining a properly sanitized environment is important for the baby's health. While cloth diapers are generally washed and reused multiple times, disposable diapers are primarily thrown away after a single use. As a baby often requires changing of a diaper more than five times per day, the ease associated with disposing of diapers and maintaining a sanitized environment is considerably important.

A number of attempts directed towards improving diapers have been made in order to simplify the disposal process of diapers. For example, U.S. Pat. No. 4,085,753 discloses a diaper having a two-ply back sheet that serves as a disposal bag or sheath within which the used diaper can be encased by turning the back sheet inside out to fully enclose the soiled diaper therewithin. U.S. Pat. No. 5,071,414 notes the awkwardness of the '753 structure, which does not allow for the diaper to be rolled up prior to disposal, and attempts to improve upon the '753 structure. With respect to the diaper disclosed in the '414 patent, a self packaging pocket is secured to the backing surface of the diaper body and forms a transverse open ended pocket to be turned inside out over the rolled up diaper after its use.

While the improvements made to disposable diapers described above are useful for their intended purpose, there remains a need for a disposable diaper that is structured to allow for easy and safe sliding of the diaper underneath the baby and into optimal changing position. The present invention provides a diaper with a hand receiving pocket that permits the installer to easily slide the diaper underneath the baby, while also serving to protect the baby from the installer's fingernails, rings, or rough skin. The hand receiving pocket of the present invention further serves as a compartment within which the diaper can be rolled up and folded into for easy disposal.

### SUMMARY OF THE INVENTION

The present invention is directed to an improved disposable diaper including a pocket in the specific area of the inside back panel of the diaper suitable for placement of the installer's hand, creating a controlled mitten effect, that allows the installer to easily slide the diaper underneath the baby's bottom and into an optimal position for installation. Insertion of the installer's hand into the pocket during the diaper changing process creates a barrier between the baby and the installer's hand, thus protecting the baby's skin from the installer's fingernails, rings, or rough hands. The pocket further serves as a means for securing a soiled diaper, which can be rolled up and folded into the pocket, allowing for easy disposal of the soiled diaper.

### BRIEF DESCRIPTION OF THE DRAWINGS

For a fuller understanding of the nature of the present invention, reference should be made to the following detailed description taken in conjunction with the accompanying drawings in which:

FIG. 1 is a topside plan view illustration showing the improved disposable diaper of the present invention, and including a pocket located on the inside back panel of the diaper for insertion of the installer's hand;

FIG. 2 is a side view illustration showing the improved disposable diaper of the present invention being directed by the installer's hand within the pocket to the optimal position for installation underneath a baby;

FIG. 3 is a perspective illustration of the improved disposable diaper of the present invention showing the front panel of a soiled diaper being folded inwards in the direction of the pocket;

FIG. 4 is a perspective illustration showing the improved disposable diaper of the present invention after the front panel of a soiled diaper has been folded and rolled into the pocket, and showing the direction that a secondary flap on the pocket is pulled to envelop the front panel within the pocket, and further showing the dual adhesive strips directed inwards to secure the soiled diaper; and

FIG. 5 is a perspective illustration showing the improved disposable diaper of the present invention after the dual adhesive strips have been attached to secure the soiled diaper.

Like reference numerals refer to like referenced parts throughout the several views of the drawings.

### DETAILED DESCRIPTION OF THE PREFERRED EMBODIMENTS

Referring to the several views of the drawings, the diaper with hand receiving pocket is shown in accordance with a preferred embodiment and is generally indicated as 10.

The diaper with hand receiving pocket 10 includes a front panel 20 having an interior face 22 that is layered with a moisture absorbent material 24 and an exterior face 26 that is made of a moisture impervious material 28. A back panel 30 has an interior face 32 that is layered with the moisture absorbent material 24 and an exterior face 34 that is made of the moisture impervious material 28. A crotch panel 36 includes an interior face 38 that is layered with the moisture absorbent material 24 and an exterior face 40 that is made of the moisture impervious material 28.

A pocket 42 is located on the interior face 32 of the back panel 30. The pocket 42 is adhered to the interior face 32 along its two sides as well as along the outer baseline 44. A secondary flap 46 is adhered to the pocket 42 along its two sides and the inner baseline 48 of the pocket 42. Adhesive strips 50, 52 extend outwards from elastic flaps 51, 53, respectively, which are attached to opposite sides of the back panel 30. The elastic flaps 51, 53 are composed of an elastomeric paper material commonly used in the art.

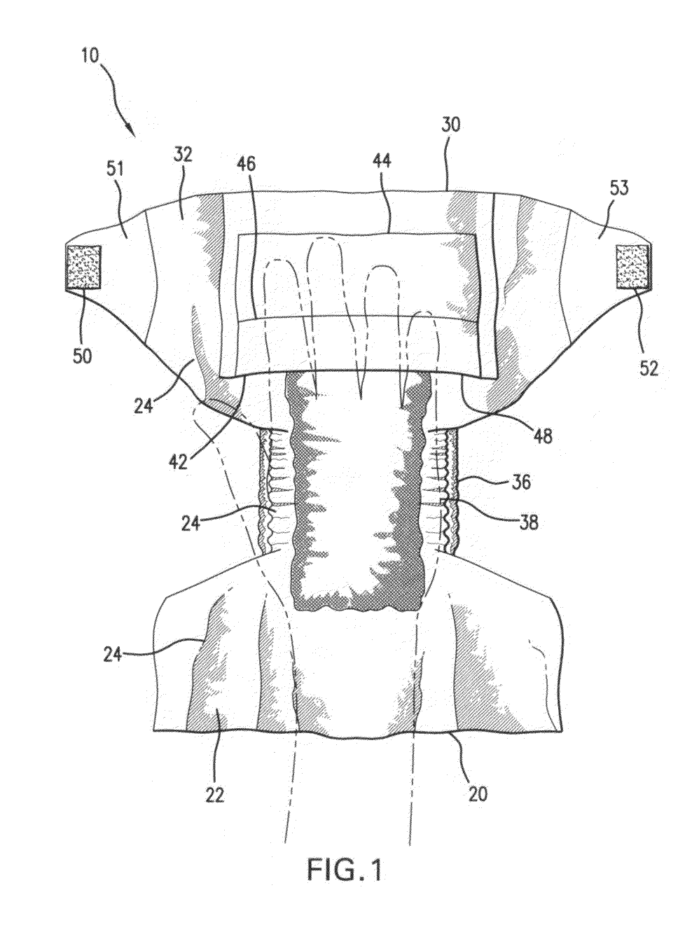

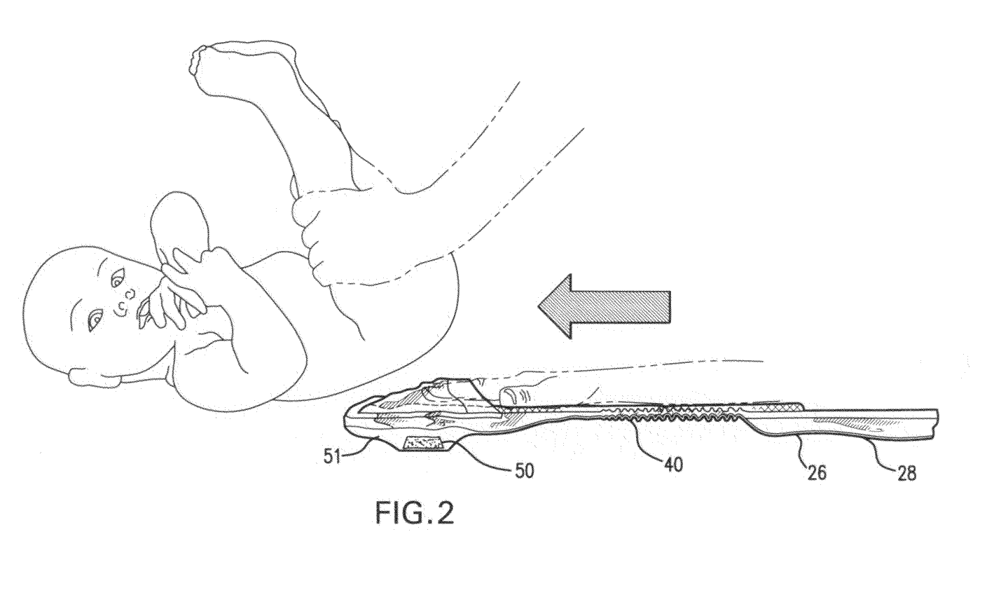

In operation, the installer slides his fingers within the pocket 42, coming into contact with the outer baseline 44 of the pocket 42. The installer's thumb remains outside of the pocket 42. The pocket 42 and the secondary flap 46 can be composed of the same elastomeric paper material used to form the elastic flaps 51, 53 to enhance the fit and elasticity associated with the pocket 42 and secondary flap 46. Referring to FIGS. 1 and 2, the palm of a properly inserted hand will be in contact with the interior face 38 of the crotch panel 36. This positioning of the hand within the pocket 42 allows the installer to easily slide the diaper 10 underneath the baby and into the optimal changing position while the installer lifts the baby's bottom up and above the changing surface by grasping the baby's legs with the opposite hand. With the hand in the pocket 42 of the diaper, the installer can hold the diaper generally flat so that at least the exterior face 34 of the back panel 30 and the exterior face 40 of the crotch panel 36 are held flat against the underlying table or counter surface. Furthermore, as the installer's hand is within the pocket 42, the baby's sensitive skin is shielded from the installer's fingernails, rings, and/or rough skin as the diaper is being positioned underneath the baby. The diaper 10 may then be secured to the baby by stretching the elastic flaps 51, 53 around the baby's waistline and attaching the adhesive strips 50, 52 to the front panel 20, forming a waistband.

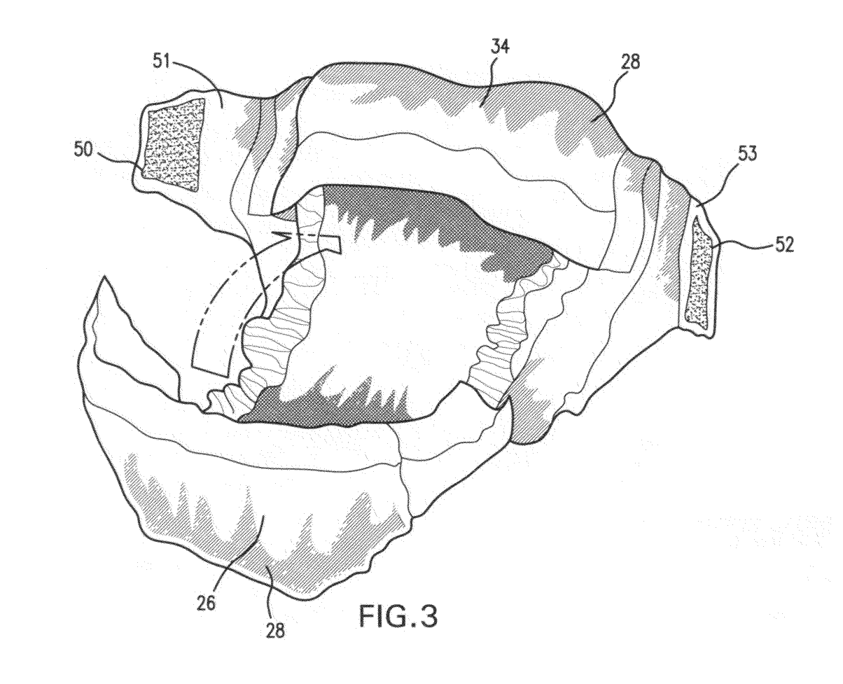

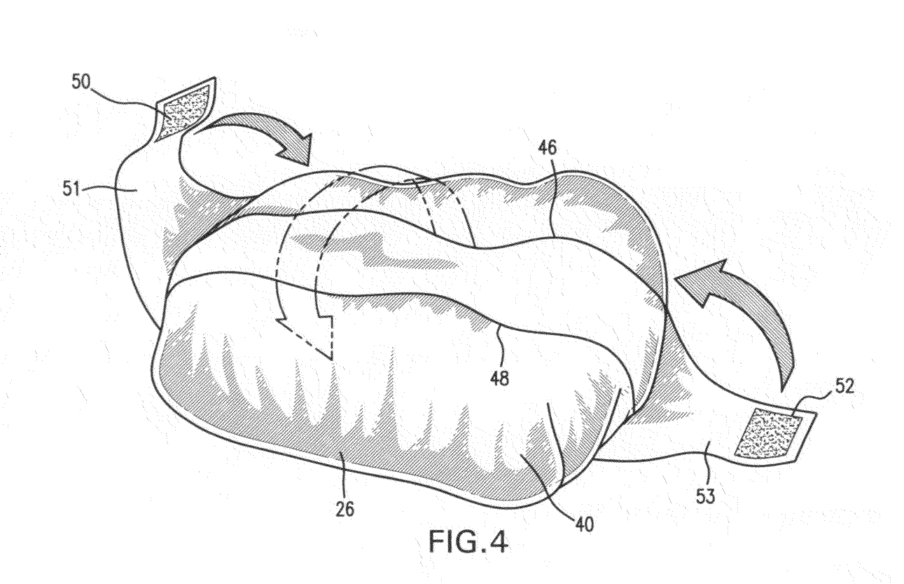

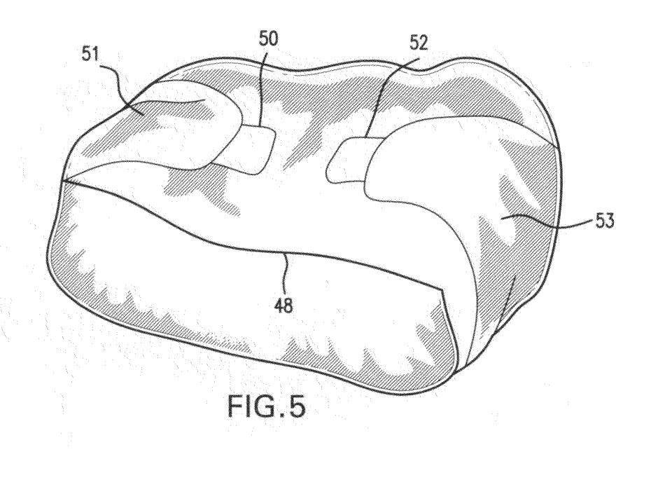

The process of disposing of a soiled diaper 10 is generally depicted in FIGS. 3, 4, and 5. After a soiled diaper 10 has been removed, the front panel 20 is rolled inwards towards the crotch panel 36. Continuing, the crotch panel 36 and the front panel 20 are rolled and folded into the pocket 42, afterwhich the secondary flap 46 is folded outwards and around the diaper 10. Lastly, the adhesive strips 50, 52 are attached to the diaper 10, securing the diaper 10 in a clean and easily disposable fashion.

While the present invention has been shown and described in accordance with several practical and preferred embodiments, it is recognized that departures from the instant disclosure are fully contemplated within the spirit and scope of the invention which is limited only by the following claims as interpreted by the Doctrine of Equivalents.

What is claimed is:

1. A diaper comprising:
a crotch panel having a top side and a bottom side, and said crotch panel including a moisture impervious exterior face and a moisture absorbent interior face;
a front panel having a moisture impervious exterior face and a moisture absorbent interior face, and said front panel being attached to the bottom side of said crotch panel;
a back panel having a moisture impervious exterior face and a moisture absorbent interior face, and said back panel being attached to the top side of said crotch panel;
a pocket located on the moisture absorbent interior face of said back panel, and said pocket having an opening facing said crotch panel, and said pocket being structured and disposed for insertion of at least a portion of an installer's hand; and
releasable fasteners on opposite sides of said back panel and being structured and disposed for releasable attachment to said front panel to secure the diaper on a baby.

2. The diaper as recited in claim 1 wherein said pocket is structured and disposed for receiving at least a portion of four figures of the installer's hand therein with the diaper held open so that the exterior face of the back panel and the exterior face of the crotch panel are held against an underlying surface.

3. The diaper as recited in claim 1 wherein said pocket is structured and disposed for enclosing said diaper after said diaper has been rolled up and folded into said pocket.

4. The diaper as recited in claim, further comprising:
a secondary flap located on said pocket having an opening opposing said crotch panel, and said secondary flap being structured and disposed for wrapping around a portion of said diaper after said diaper has been rolled up and folded into said pocket.
